(19) United States
(12) Patent Application Publication
(10) Pub. No.: US 2024/0058523 A1
(43) Pub. Date: Feb. 22, 2024

(54) AUTOINJECTOR WITH A SQUEEZE ARRANGEMENT

(71) Applicant: Sanofi, PARIS (FR)

(72) Inventors: Ramsay Black, Melbourn, Hertfordshire (GB); Uwe Dasbach, Frankfurt am Main (DE); Thomas Mark Kemp, Melbourn, Hertfordshire (GB); William Timmis, Melbourn, Hertfordshire (GB)

(21) Appl. No.: 18/267,037

(22) PCT Filed: Dec. 16, 2021
(86) PCT No.: PCT/EP2021/086109

§ 371 Date: Jun. 13, 2023

(30) Foreign Application Priority Data
Dec. 17, 2020 (EP) 20315495.0

(51) Int. Cl. A61M 5/142; A61M 5/20

(52) U.S. Cl. CPC A61M 5/14232; A61M 5/2033; A61M 2205/103; A61M 2205/8281

(57) ABSTRACT

The invention relates to an autoinjector comprising a housing (125) which is configured to receive a reservoir (120) with a fluid, a pump arrangement (100), which is configured to drive the fluid from the reservoir (120) towards and through an outlet (180) of the autoinjector in a dispensing operation.

### FIELD OF INVENTION

The present disclosure relates to an autoinjector.

### BACKGROUND OF INVENTION

In regular drug delivery devices, where a single drive mechanism which may be housed in a housing of the drug delivery device is used in conjunction with several cartridges, syringes or ampules to dispense drug contained in the cartridge, syringe or ampule from the device.

Devices of this kind, however, are designed for housing a cartridge and a syringe, and due to the shape of these components the overall shape of the device is adapted to the syringe, cartridge or ampule, as they are rigid components. This is usually decisive or at least limiting for the form factor of the device.

### SUMMARY

It is an object of the present disclosure to provide an alternative autoinjector. This object is solved by the present disclosure and, particularly, by the subject-matter of the independent claim. Advantageous embodiments and refinements are subject to the dependent claims.

The disclosure relates to an autoinjector comprising a housing, which is configured to receive a reservoir with a fluid, e.g. a liquid, and a squeeze arrangement, which is configured to drive the fluid from the reservoir towards and through an outlet of the autoinjector in a dispensing operation.

Using a squeeze arrangement for driving the fluid from the reservoir to the outlet has the advantage that the amount of fluid, which can be moved and delivered through the needle, can be controlled by the squeeze arrangement. Also there is some degree of freedom to choose the shape of the reservoir. For example it can have a different shape than a syringe.

In an embodiment the squeeze arrangement comprises a movable element which mechanically interacts with the reservoir such that a movement of the movable element causes a squeezing of the reservoir to drive at least a portion of the fluid from the reservoir towards the outlet.

In an embodiment the squeeze element comprises a or is a roller, which is configured to rotate around a roller axis while it is rotating at the same time around the rotation axis and thereby squeezing the reservoir. The rolling function of the roller leads to a reduced resistance caused by friction between roller and reservoir while the roller is moved around the rotation axis. The roller axis is the main axis of the roller and is arranged with an angle in relation to the rotation axis.

In an embodiment the roller comprises a shape of a cylinder or a cone, wherein the cone widens radially outwardly relative to the rotation axis.

In an embodiment the housing may have a shape with a base which has a larger diameter than the height, which extends along the rotation axis. In an embodiment the shape comprises a cylinder, in particular a cylinder with rounded edges.

In an embodiment the reservoir is arranged circumferentially around the rotation axis, wherein the reservoir is oriented along a circle segment, such that the fluid contained in the reservoir can be driven towards the outlet with a rotation of the movable element which comprises less than a full revolution around the rotation axis. This indicates a simple construction and the amount of fluid to be released relates to the amount of fluid in the reservoir. Further it requires less energy for releasing all fluid from the reservoir to the needle compared to a system where the movable element requires several revolutions.

In an embodiment the reservoir comprises a pouch or another hollow shaped body which is configured for moving fluid through it. The pouch can be collapsible.

In an embodiment, the reservoir comprises a flexible material, such as a flexible plastic which is collapsible, which means the reservoir may be non-elastically deformable. That is to say, when deformed, the reservoir remains in its deformed shape. The pouch may comprise more than one material which may be specific adaptable to its inside and outside according to chemical and/or mechanical requirements. For example regarding the outside of the pouch the material may be mechanically robust with regard to the pressure applied to the pouch by squeezing. Regarding the inside of the pouch a material may be required which does not engage in a chemical reaction with the fluid contained in the pouch.

Employing a flexible reservoir has the advantage of improved robustness, in particular compared to a glass syringe which is fragile and can break. Further, there is an improved drug integrity and less contamination risk, because the reservoir only comprises one opening, which needs to be sealed, which is on the injection side. In autoinjectors with syringes, for example, there is additionally the side of the stopper to be sealed.

Another advantage is the opportunity for a different form with usability benefits, because the reservoir can be adjusted to the form of the device. Its compactness and consistency avoids large pre-filled syringe (PFS) tolerances reducing injection variability. In particular it is possible to employ plastic instead of glass for the reservoir which can be manufactured with a higher precision compared to glass. It further has a reduced stalling risk and no stopper friction, because there is no stopper required. The reservoir can be filled by vacuum filling to eliminate any air, or steam purging prior to container closure. In the case of vacuum filling the pouch can be pulled apart (e.g. by a vacuum) which creates a vacuum inside the pouch. This pulls liquid from a connected container inside the pouch.

In an embodiment the outlet comprises a needle.

In an embodiment the autoinjector comprises a spring comprising a spring portion which is configured to rotate around a rotation axis and which is mechanically coupled to the movable element, such that it moves the movable element around the rotation axis. In this way the release of the fluid through the reservoir and the needle is to some extent automatized and therefore easy to use for a patient. The spring may be biased when it is assembled along with the movable element. The spring may be activated with a trigger so its stored potential energy translates into kinetic energy, thereby driving the movable element. The movable element in turn drives the fluid from the reservoir to the needle. Depending on the configuration of the spring with respect to the stored energy the overall distance of the movement of the spring and the movable element can be determined. By predetermining the distance of movement of the movable element the amount of fluid which can be driven through the reservoir and through the needle can be indirectly predetermined. In case the fluid relates to a medicament, the dose of the medicament can be adjusted, for example by the size of the reservoir. These adjustments can be done in the process of assembling the device. Once the device is assembled the amount of dose will be released which is provided by the configuration.

The spring may support the movable element. For example, one portion of the spring may extend into the movable element. The roller axis may run through and/or parallel to this portion of the spring.

In an embodiment the spring comprises a torsion spring which is configured to move the movable element around the rotation axis such that the fluid contained in the reservoir is moved towards the outlet within one revolution of the movable element around the rotation axis or less. A torsion spring is easy to control, it has a compact shape and the mechanical energy which it can release can be predetermined.

In an embodiment the housing comprises a supporting element, wherein the reservoir is arranged between the supporting element and the movable element such that when the reservoir is squeezed by the movable element the reservoir is pressed towards the surface of the supporting element. This not only ensures that a portion of the fluid is moved, but it also ensures that the force of the movable element towards the reservoir is reliably transferred into squeezing and in consequence in moving the fluid through the reservoir. The supporting element may comprise a plate with a flat face.

The supporting element may comprise or may be a protrusion protruding from a bottom of the housing towards the reservoir. The supporting element or the protrusion, respectively, may extend along a curve parallel to the curve along which to the reservoir extends. For example, the supporting element is arranged along a circle element which is parallel to the circle element along which the reservoir is arranged.

A width of the supporting element, e.g. measured at the highest point of the protrusion, may be smaller than the width of the reservoir. The width of the reservoir is, e.g., the maximum width of the reservoir.

In an embodiment the autoinjector comprises a delivery tube which is in fluid communication with the reservoir, and which is arranged between the reservoir and the outlet such that the delivery tube supplements fluid to the outlet which is provided by the reservoir during a dispensing operation.

The reservoir expediently has a greater diameter than the delivery tube. The larger amount of fluid and the larger diameter of the reservoir lead to a fluid pressure from the reservoir to the delivery tube. A continuous flow of fluid can be established from the reservoir to the delivery tube as soon as fluid is moved out of the delivery tube. This is of importance when a suspension of a medicament is undertaken where a continuous flow of liquid is essential, and at the same time a predetermined dose needs to be ensured.

In an embodiment the reservoir comprises a narrowing portion which connects the reservoir and the delivery tube. The narrowing portion ensures that the fluid is forced into the delivery tube, and the amount of fluid which is not released from the reservoir is reduced to a minimum.

In an embodiment the autoinjector comprises an outlet drive mechanism comprising
an outlet,
an interface element which is connected to or integrated with the outlet, wherein the interface element is movable from a first axial position to a second axial position along the rotation axis,
a trigger which is operatively connected to the interface element wherein the trigger is movable from a first trigger position to a second trigger position along the rotation axis, wherein
   in the first trigger position the interface element is releasably locked from moving from the first axial position to the second axial position, and
   in the second trigger position, the interface element is movable to the second axial position, wherein
   the movement of the trigger from the first trigger position to the second trigger position causes the interface element to be released from the first axial position such that the interface element is movable to the second axial position.

The outlet may comprises a needle. The interface element may comprise at least one interface feature. The interface element may comprise a needle holder. The interface feature may comprise a needle holder surface which is arranged at the needle holder and which is oriented rectangular with respect to the rotation axis. Preferably the needle holder comprises a needle holder ledge, wherein the needle holder surface is arranged at the needle holder ledge. The needle holder may comprise two needle holder ledges which are arranged opposite to each other with respect to the rotation axis, and each comprising a needle holder surface. The needle holder may comprise a cylindrical body wherein its axis is the rotation axis. The two needle holder ledges are arranged on the radial outside of the cylindrical body of the needle holder. The needle holder may comprise mechanical guides, for example axial grooves, on the radial outside of the cylindrical body for guiding a movement of trigger along the rotation axis.

The trigger may comprise a button, one ore more trigger arms and one or more trigger interfaces. The trigger arms may extend from the button. Preferably, the trigger comprises two trigger arms. The trigger interfaces are arranged at the endings of the trigger arms. The trigger interfaces may comprise oblique trigger surfaces.

When the trigger moves along the rotation axis the trigger arms may be guided by the mechanical guides of the needle holder, wherein the mechanical guides of the needle holder secure the trigger arms against rotation. It is also possible that the trigger arms are secured against rotation by a base element which comprises mechanical guidance and which is fixed to or integrated with the housing of the device. The base element may comprise a base element main body which is fixed to or integrated with the housing. The base element main body may comprise a hole which may be arranged on the rotation axis and which is configured so that the needle is movable through the hole along the rotation axis. The hole is may be sealed by a sealing which seals hole and the housing towards the outside. The sealing may be penetrated by the needle when the needle is moved by the drive spring in the direction of the hole. The base element may comprise base element arms which extend from the base element main body towards the inside of the housing of the device. The base element arms are arranged around the rotation axis and are spaced apart with a spacing. The spacings between the base element arms are configured to receive the radial outward part of the trigger arms for guiding them axially in the assembled state. In the assembled state the needle holder ledges are received in the other spacings between the base element arms and are secured against rotation with respect to the base element and because the base element is fixed to or integrated with the housing the needle holder ledges and the needle holder is secured against rotation with respect to the housing.

The outlet drive mechanism is enables the release of the needle to move to a position for injection when a trigger is moved from a first trigger position to a second trigger position. In order to avoid an accidental movement of the needle the needle holder is locked to a first axial position until the trigger is moved to its second trigger position.

In an embodiment the autoinjector comprises a holding element which is in mechanical contact with the interface element, wherein
the holding element is rotatable around the rotation axis relative to the interface element from a blocking position to a release position, wherein
in the blocking position the interface element is releasably locked by the holding element to move from the first axial position to the second axial position, and
in the release position the interface element is movable from the first axial position to the second axial position, wherein
the movement of the trigger from the first trigger position to the second trigger position causes the holding element to rotate from the blocking position to the release position.

The holding element may comprise at least one first holding interface which is configured to interact with one of the trigger surfaces. The holding element may comprise a collar. The collar comprises one ore more collar trigger arms, preferably two trigger arms, which are arranged opposite to each other with respect to the rotation axis. The one or more collar trigger arms comprise first holding interfaces. The first holding interfaces may comprise first holding surfaces which may be oblique holding surfaces, and which face the trigger interfaces which may be oblique trigger surfaces along the rotation axis. The oblique trigger surfaces are configured to interact with the oblique holding surfaces. The oblique trigger surfaces and the oblique holding surfaces are configured and oriented to slide on top of each other. When the trigger is moved from the first trigger position to the second trigger position the oblique trigger surfaces and the oblique holding surfaces get in mechanical contact such that the oblique trigger surfaces are pushed on the oblique holding surfaces. When the surfaces slide on top of each other the axial movement of the trigger arms and the oblique trigger surfaces, which apply axial forces to the oblique holding surfaces and the collar trigger arms, causes the collar trigger arms and the collar to rotate around the rotation axis. The rotation of the collar is relative to the needle holder and the needle holder ledges which are secured against rotation.

It is also possible that only one of the trigger surfaces and the holding surfaces is oblique.

The holding element may further comprise at least one second holding interface which is configured to interact with the interface element, in particular with the interface feature, which may be a surface of the needle holder ledge. The holding element may comprise one or more collar holding arms, preferably two holding arms, which are arranged opposite to each other with respect to the rotation axis. Each of the collar holding arms comprise a second holding interface which may comprise a second holding surface. Each of the second holding surfaces face an interface feature which may be needle holder surface. In the first axial position of the needle holder the needle holder surfaces are in mechanical contact with the second holding surfaces such that when the collar holding arms rotate relative to the needle holder the needle holder surfaces slide on top of the second holding surfaces. Thereby the second holding surfaces block the needle holder surfaces from moving in the axial direction.

The one ore more collar trigger arms and the one or more collar holding arms are arranged alternating around the rotation axis, wherein each neighbouring collar trigger arm and collar holding arm are separated by a spacing. The spacings are configured to receive the needle holder ledges and/or the trigger arms.

In an assembled state of the outlet drive mechanism the collar is arranged inside the volume which is enclosed by the base element arms, such that the collar can rotate inside the base element.

When the trigger is moved from the first trigger position to the second trigger position the applied force to the collar trigger arms causes the collar trigger arms to rotate relative to the needle holder and the needle holder ledges. The rotation continues until the needle holder ledges face the spacings of the collar.

Further the needle holder ledges are blocked by the collar trigger arms against axial movement by the collar holding arms in the first axial position of the needle holder. Further, in the release position of the holding element the drive spring moves the needle holder to the second axial position. The blocking and release position of the collar are only different in rotation/angle but not in the axial position.

The trigger interface is further configured to translate an axial movement of the trigger and its interface into a rotational movement a holding element. The trigger interface may comprise arms which extend along the rotation axis, such that they can mechanically interact with the collar.

The collar provides an additional safety with regard to an accidental movement of the needle. The needle holder is only released when the collar has undertaken a rotational movement which is dependent to the axial movement of the trigger. The needle holder cannot rotate on its own because it is secured against rotation. Further in the blocking position of the collar the surfaces of the collar holding arms are pressed towards the surfaces of the needle holder ledges by the compressed drive spring. This ensures that the collar cannot rotate accidentally.

In an embodiment the autoinjector comprises an outlet drive unit which is operatively connected to the trigger and to the interface element, wherein
the outlet drive unit being configured to provide energy for moving the interface element from the first axial position to the second axial position, wherein the outlet drive unit has a first drive unit state and a second drive unit state, wherein
in the first drive unit state the outlet drive unit has energy stored and the interface element is in the first axial position and the holding element is in the blocking position and the interface element is prevented from moving to the second axial position, and
in the second drive unit state the outlet drive unit can transfer energy to the interface element so that the interface element is moved the first axial position to the second axial position along the rotation axis when the holding element is in the release position, wherein
the movement of the trigger from the first trigger position to the second trigger position causes the outlet drive unit to change from the first drive unit state to the second drive unit state.

The outlet drive unit may comprise a drive spring. The drive spring may be arranged between the trigger and the needle holder along the rotation axis. When the needle holder is in the first axial position, the drive spring is in the first drive unit state where the drive spring is compressed. When the needle holder is released so that it can move to the second axial position the drive unit is in the second drive unit state where the drive spring can expand and transfer mechanical energy to the needle holder. Under the force of the expanding drive spring the needle holder moves to its second axial position along the rotation axis.

When the release of the needle holder is initiated by the trigger the needle holder is then moved by the drive spring to the second axial position which can be the position for injection and dispension. In this way the trigger initiates an injection which occurs automatically and is driven by the drive spring once the trigger has initiated the release of the needle holder.

In an embodiment the trigger may also cause the dispension of the fluid.

In an embodiment the housing may has a shape with an overall base which has a larger diameter than the height, which extends along the longitudinal axis. In an embodiment the shape comprises a cylinder, in particular a cylinder with rounded edges.

In an embodiment the autoinjector comprises the reservoir with a medicament or drug.

In an embodiment the autoinjector is disposable or a single-use device, for providing a single dose.

### BRIEF DESCRIPTION OF THE DRAWINGS

Embodiments will now be described, by way of example only, with reference to the accompanying drawings, in which:

FIGS. 1A to 1C show schematic top-views from different angles of the autoinjector.

FIG. 2 shows a cross-section from a side-view of the autoinjector.

FIG. 3A shows a cross-section from a side view of the squeezing arrangement.

FIG. 3B shows a cross-section from an angled top view of a squeezing arrangement.

FIG. 4A shows an exploded view of the needle drive mechanism.

FIG. 4B shows a view of the needle drive mechanism with the drive spring in a first state and assembled with a base element.

FIG. 4C shows a view of a needle drive mechanism with a drive spring in a first state.

FIG. 4D shows a view of the needle drive mechanism with the drive spring in a second state.

### DETAILED DESCRIPTION

The same reference numbers apply to the same features throughout the figures and the following explanations.

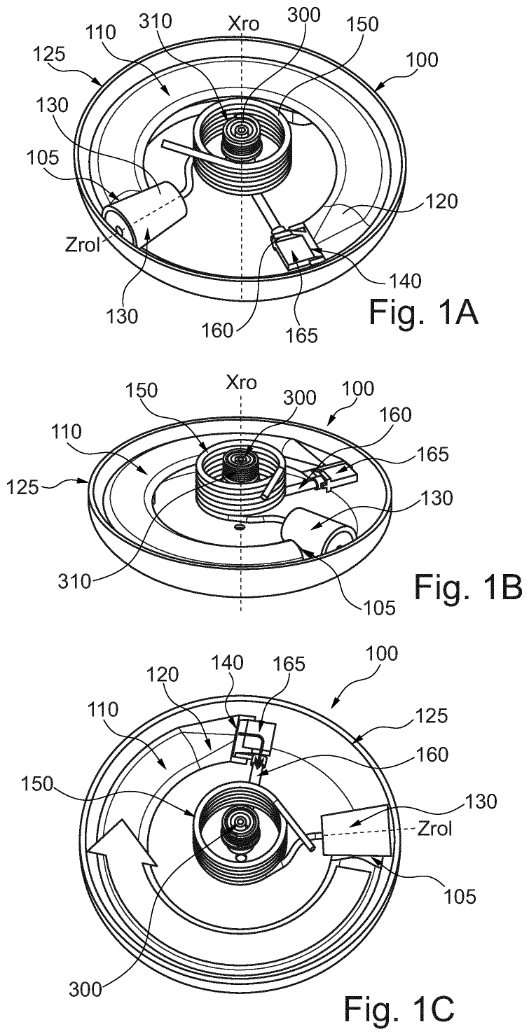

FIG. 1A to 10 show schematic top-views from different angles of the components of the autoinjector. As an example regarding the FIGS. 1A to 10 in the following FIG. 1A is described.

FIG. 1A shows a top view of an embodiment of the autoinjector 100. It shows a squeeze arrangement 105. A roller 130 is arranged rotatable around a rotation axis Xro. During a rotation the roller 130 also rotates around its own roller axis Zrol, which is arranged with an angle towards the rotation axis Xro. The roller 130 rolls in a direction opposite to the rotation around the rotation axis Xro. The roller 130 is configured to squeeze a pouch 110, which comprises a fluid material, e.g. a liquid medicament or medicament formulation. The pouch 110 is arranged around the rotation axis Xro such that it comprises a cross-section of a circle segment. The pouch 110 is arranged in a radial distance from the rotation axis Xro so that the roller 130 can squeeze the pouch 110 during rotation. One ending of the pouch 110 is in fluid communication with a needle 180. This ending comprises a narrowing portion 120, which is in fluid communication with a delivery tube 160. This delivery tube 160 is in fluid communication with the needle 180. There can be arranged and additional spout 165 which connects the delivery tube 160 and the narrowing portion 120. The spout 165 enables a coupling between the pouch 110 and the delivery tube 150. The spout 165 has different material properties than the pouch 110. For example it is more stiff so that the alignment of the pouch 110 in the housing 170 is based on the position and orientation of the spout 165. The delivery tube 160 is extending radially inward from the pouch 110 where it connects with the needle 180. The needle 180 is arranged in the interior of the spring 150 and extends along the rotation axis Xro. The fluid which is squeezed by the roller 130 moves within the pouch 110 further to the narrowing portion 120, then via the delivery tube 160 to the needle 180. The fluid of the pouch 110 can be released through the needle 180.

The roller 130 squeezes along the entire length of the pouch 110. It is also possible that the roller 130 stops before the entire length of the pouch 110 is squeezed. However, in this case at least a portion of the narrowing part 120 is squeezed. As the pouch 110 is arranged such that it comprises an overall cross-section of a circle segment the length which is squeezed by the roller 130 corresponds to the length of this circle segment. Overall the roller 130 squeezes a length which is less than a full circle. Once the roller 130 has squeezed all the length of the circle segment it is stopped by a roller stopper 140 which is arranged at the ending of the narrowing portion 120. In this way the fluid within the pouch 110 is removed by the squeezing of the roller 130 within the circumferential length of the circle segment. Further the roller 130 has a predetermined stop and an uncontrolled movement can be avoided. The roller stopper 140 can be a block with a rectangular cross-section comprising metal or plastic. The roller stopper 140 may be mounted on the housing 125.

The pouch 110 is arranged at a plate 170 which comprises a flat face towards the pouch 110, such that during a squeezing operation the pouch 110 is arranged between the plate 170 and the roller 130. During a squeezing operation the roller 130 presses the pouch 110 towards the plate 170. In this situation the plate 170 supports the pouch 110 and ensures that the force which is applied from the roller to the pouch 110 is transferred to the movement of the liquid inside the pouch 110.

The plate 170 can be mounted inside the housing 125. The plate 170 and the housing 125 can be also made in one piece or they can be separate parts.

The autoinjector 100 further comprises a spring 150 which is mechanically connected to the roller 130 and drives the roller 130 around the rotation axis Xro, and thereby moves itself around the roller axis Zrol. The spring 150 is preferably biased during assembly, so that by triggering the spring 150, for example by a user, the required energy for the squeezing and dispensing operation is released in order to drive the roller 130 around the rotation axis Xro. The spring can be a torsion spring 150. The spring 150 is arranged around the rotation axis Xro, within circle segment of the pouch 110.

The roller 130 may have a cylindrical or conical shape. The pouch 110 can be a pouch, in particular a collapsible pouch, so that after the squeezing, where the pouch 110 was deformed it does not undeform, but remains in the deformed state.

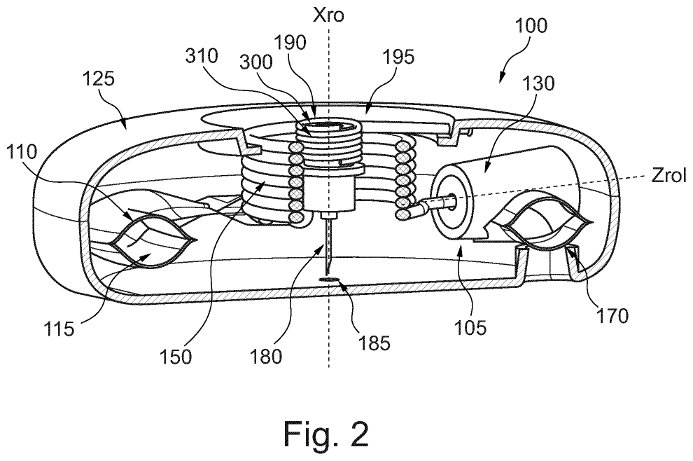

FIG. 2 shows a cross-section from a side-view of the autoinjector. The squeezing arrangement 105 is arranged inside the housing 125. The housing 125 may comprise two parts which are joined together. The housing 125 comprises a needle opening 185, so that the needle can at least partially move along the rotation axis Xro to be extended from the housing 125, preferably from a position, where it was completely retained in the housing. For example in the case the autoinjector is used for an injection and pressed on the skin of a patient, the needle 180 can directly be pierced from the autoinjector through the needle opening 185 into the skin. The needle opening 185 needs to be sealed, such that the needle 180 fulfils the requirements of cleanliness for needles 180 for medicine applications. The movement of the needle 180 can be triggered via a trigger opening 190, which is arranged at the housing 125. The user or the patient of the autoinjector may trigger the injection by pressing a button (not shown here) through the trigger opening 190 and thereby initiating a movement of the torsion spring 150 or the needle 180 by the needle drive mechanism 300.

The housing 125 has shape of a cylinder with rounded edges. The rounded edges are of advantage in order to avoid injuries at a patient's skin due the sharp edges. The rotation axis Xro comprises the cylindrical axis.

The housing 125 also comprise a window 195 for monitoring the progress of a squeezing operation and indicating the end of the squeezing operation or the end of the injection.

The autoinjector is disposable or a single-use device, for providing a single dose.

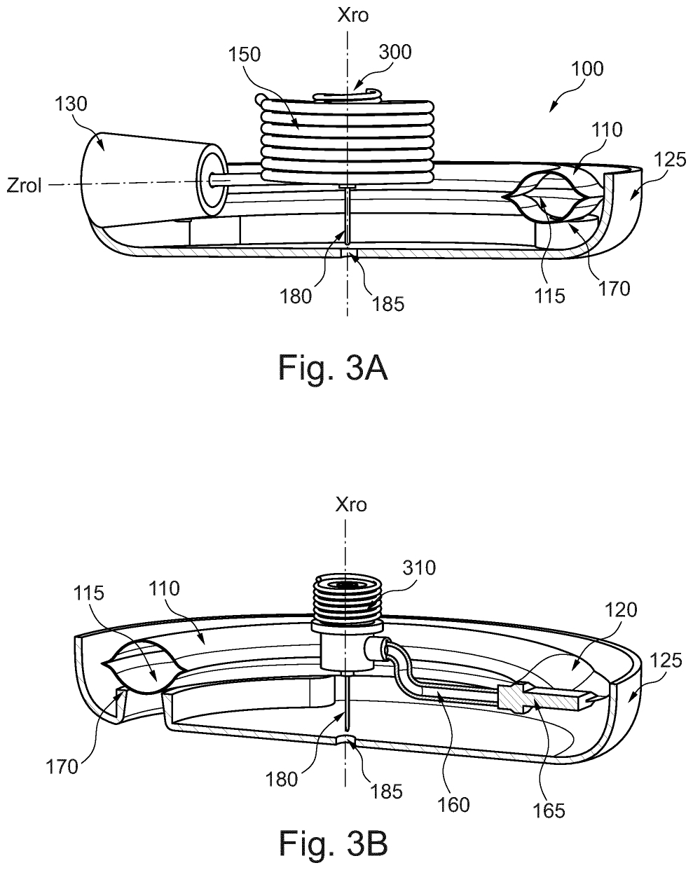

FIG. 3A shows a cross-section from a side view of the autoinjector. In addition to the previous Figures it is shown the pouch cross-section 115, which can be elliptically shaped. It is also possible that the pouch 110 is made of two parts of material which are bonded together. These two parts may comprise all the length of the pouch 110. In case the pouch cross-section is elliptically shaped the longer axis of the ellipsis crosses the area of the pouch 110 where the two parts are bonded together. A pouch 110 with an elliptically shaped pouch cross-section 115 has the advantage that its width is larger than its height, so that a roller 130 which has a cylindrical or conical shape can squeeze the pouch 110 more efficiently compared to circular pouch cross-section 115 with a larger height.

FIG. 3B shows a cross-section from an angled top view of a reduced squeezing arrangement without the torsion spring 150.

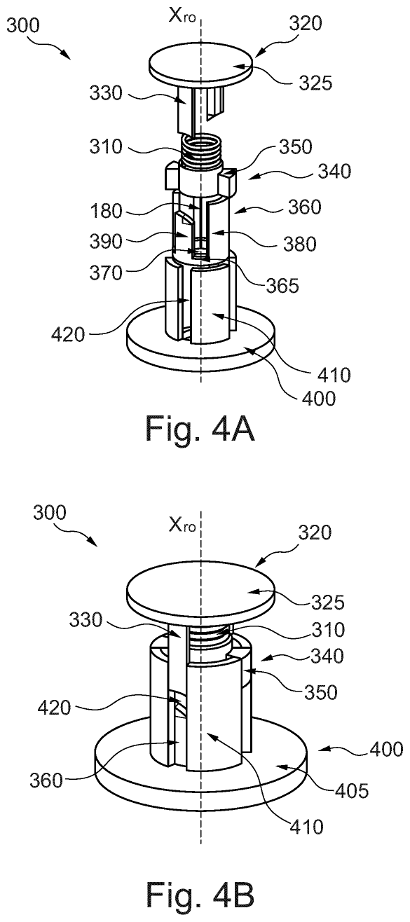

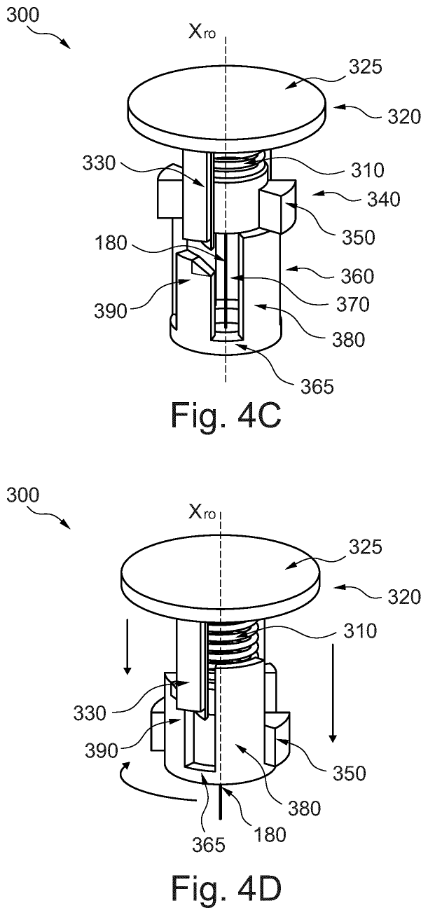

FIG. 4A shows an exploded view of a needle drive mechanism 300 in detail.

The needle drive mechanism 300 comprises a trigger button 320, a drive spring 310, a needle holder 340, a collar 360 and a base element 400. The needle 180 is mechanically connected to the needle holder 340.

The trigger button 320 comprises a trigger button main body 325 and two trigger button arms 330 extending from the trigger button main body 325 along the rotation axis Xro. The trigger button main body 325 comprises a cylindrical shape wherein the height or thickness is smaller than the diameter forming an overall disc-like shape. The trigger button main body 325 can also comprise any other shape such as a rectangular or squared thin plate. The trigger button main body 325 may be connected to or integrated with the housing 105 of the device and then may have the same thickness as the housing 105. The two trigger button arms 330 are arranged opposite to each other with respect to the rotation axis Xro. Each of the trigger button arms 330 comprises a radial outward part and a radial inward part. Each of the trigger button arms 330 comprises an oblique surface on the ending away from the trigger main body 325 with respect to the rotation axis Xro. The oblique surfaces are arranged on the radial inward part.

The drive spring 310 is arranged between the trigger button main body 325 and the needle holder 340 along the rotation axis Xro and is expandable and compressible along the rotation axis Xro. The collar 360 can be arranged inside the base element 400. When the drive spring 310 is compressed the needle holder 340 is arranged between the collar 360 and the drive spring 310.

The needle holder 340 comprises a main body with a cylindrical shape and two needle holder ledges 350 which extend radially outward from the main body and are arranged on opposite sides with respect to the rotation axis Xro. The needle holder 340 is operatively connected to the drive spring 310, such that when the drive spring 310 expands along the rotation axis Xro the needle holder 340 and the needle 180 move along the rotation axis Xro away from the trigger button main body 325. The needle holder ledges 350 can have the shape of a wedge, wherein their cross-section decreases radially inwardly.

The collar 360 comprises a collar base 365, two collar holding arms 380 and two collar trigger arms 390. The two collar holding arms 380 and the two collar trigger arms 390 each extend from the collar base 365 towards the trigger button main body 325 along the rotation axis Xro. The two collar holding arms 380 are arranged on opposite sides with respect to the rotation axis Xro. The two collar trigger arms 380 are arranged on opposite sides with respect to the rotation axis Xro. The collar holding arms 380 and the collar trigger arms 390 are arranged alternating around the rotation axis Xro and are spaced apart by collar spacings 370. The collar holding arms 380 comprise a rectangular shape. The collar trigger arms 390 also comprise a rectangular basis-shape but with an oblique surface at their endings directing away from the collar base 365 and facing the oblique surfaces of the radial inward part of the trigger button arms 330. It is also possible that wither the trigger button arms 330 or the collar trigger arms 390 comprise oblique surfaces. For example when the only trigger button arms 325 comprise oblique surfaces, a movement along the rotation axis Xro would still lead to a rotation of the collar 360 when the oblique surface pushes on an edge of a collar trigger arm and thereby further moving along the rotation axis Xro. The collar base 365 comprises a hole such that the needle 180 can move through the hole along the rotation axis Xro.

The base element 400 comprises a base element main body 405 and four base element arms 410 which are each extending from the base element main body 405 in the direction towards the trigger button main body 325 along the rotation axis Xro. The base element main body 405 comprises a cylindrical shape wherein the height or thickness is smaller than the diameter forming an overall disc-like shape. The base element main body 405 can also comprise any other shape such as a rectangular or squared thin plate. The base element main body 405 may be connected to or integrated in the housing 105 of the device. The base element arms 410 extend from the base element main body 405 along the rotation axis Xro and are arranged around the rotation axis Xro, thereby enclosing a volume, which is configured to receive the collar 360 when it the needle drive mechanism 300 is assembled. The base element arms 410 are separated towards each other by spacings 420. Two of the spacings 420 are configured to receive the radial outward part of the trigger button arms 330 such that the trigger button arms 330 are axially guided within the spacings 420 but secured against rotation. The other two spacings are configured to received the needle holder ledges 350 along the rotation axis Xro which are then secured against rotation. The base element main body 405 comprises a hole such that the needle 180 can move through the hole along the rotation axis Xro to the outside of the device for injection. The hole can be sealed with a sealing, which can be penetrated by the needle.

FIG. 4B shows a view of the needle drive mechanism 300 in an assembled state. The drive spring 310 is compressed between the trigger button 320 and the needle holder 340 with respect to the rotation axis Xro, so that the drive spring 310 biases the needle holder 340. The trigger button arms 330 have entered the base element spacings 420 partially so that they can move along the rotation axis Xro wherein they are guided by the base element arms 410 which are forming the base element spacings 420. When the trigger button arms 330 are axially movable inside the base element spacings they are secured against rotation by the base element arms 410.

Alternatively the trigger button arms 330 could be also secured against rotation by the needle holder 340. For the cylindrical body of the needle holder 340 may comprise an additional groove which is oriented along the rotation axis Xro, and which is configured to mechanically guide the trigger button arms 330 along the rotation axis Xro, thereby securing them against rotation.

The needle holder ledges 350 are in the same way arranged in base element spacings 420. In this way the needle holder ledges 350 are secured against rotation by the base element 400 and if the base element 400 is a part of the housing 105 of the device the needle holder ledges 350 and the needle holder 340 are secured against rotation by the housing 105. Further the needle holder ledges 350 are axially blocked by the collar 360. The collar 360 is arranged inside the space which is enclosed by the base element arms 410.

The needle holder 340 comprises a hole for receiving a pipe (not shown). The hole can be directed radially inwardly from the needle holder ledge 350. The pipe is in fluid communication with the needle 180 and the delivery tube 160. In this way the fluid of the pouch 110 can be transferred to the needle 180 for dispension.

FIG. 4C shows a view of the assembled needle drive mechanism 300 without the base element 400 where the drive spring 310 is compressed between the trigger button 320 and the needle holder 340 with respect to the rotation axis Xro, so that the drive spring 310 biases the needle holder 340. The needle holder 340 is arranged between the drive spring 310 and the collar 360 with respect to the rotation axis Xro. The needle holder ledges 350 are in mechanical contact with the ending surfaces of the collar holding arms 380, such that the force of the compressed drive spring 310 acts on the collar holding arms 380 through the needle holder ledges 350. The trigger button arms 330 are arranged radially outward with respect to the drive spring 310 and the needle holder 340. The endings of the trigger button arms 330 are facing the endings of the collar trigger arms 390 but are spaced apart axially. Both the endings of the trigger button arms 330 and the endings of the collar trigger arms 390 comprise an oblique surface, such that when the oblique surface of a trigger button arm 330 is pressed towards the surface of a collar trigger arm 390, the surfaces slide towards each other, and the additional movement along the rotation axis Xro by the trigger button arms 330, which applies a force to the collar trigger arms 390, causes the collar trigger arms 390 and the collar 360 to rotate.

FIG. 4D shows a view of the assembled needle drive mechanism 300 without the base element in which the drive spring 310 is expanded along the rotation axis Xro. The needle holder 340 has been moved along the rotation axis Xro in a direction away from the trigger button main body 325 due to the force of the expanding drive spring 310. Thereby, the needle holder ledges 350 have engaged into the collar spacings 370, thereby guiding the movement of the needle holder 340 along the rotation axis Xro.

When the needle drive mechanism 300 is in the status as shown in FIG. 4B or 4C, i.e. when the drive spring 310 is compressed and the needle holder 340 is blocked from moving in the direction away from the trigger button main body 325, and the trigger button 320 is pressed in a direction towards the drive spring 310 along the rotation axis Xro the trigger button arms 330 are moved towards the collar trigger arms 390. When the oblique surfaces of the trigger button arms 330 are pressed towards the surfaces of the collar trigger arm 390, the axial movement of the trigger button arms 330 cause a rotational movement of the collar trigger arms 390 and the collar 360. The rotation of the collar 360 is relative to the needle holder ledge 350. Because of the rotation of the collar 360 also the collar holding arms 380 rotate relative to the needle holder 340. During this rotation the needle holder ledges 350 are biased in the axial direction towards the collar holding arms 380 by the drive spring 310. The rotation of the collar holding arms 380 continues until at the collar spacings 370 face the needle holder ledges 350. At this point the needle holder ledges 350 are no longer blocked by the collar holding arms 380 and the trigger button arms 330 have disengaged from the collar trigger arms 390. The needle holder 340 then can axially move in a direction away from the trigger button main body 325. The collar holding arms 380 are longer than the collar trigger arms 390. It is also possible that the collar holding arms 380 are of the same length as the collar trigger arms 390. In this case the trigger button arms 330 needed to be adjusted in their lengths. The needle holder ledges 350 then engage in the collar spacings 370 and the needle holder 340 moves together with the needle 180 in a direction away from the trigger button main body 325 towards the base element 400 and further to the injection site under the force of the expanding drive spring 310.

The device may have a height between 10-40 mm, and in particular a height between 15-30 mm. The base of the device may have a diameter between 45-90 mm, and in particular a diameter between 50-70 mm. In particular the height of the device may be smaller by a factor of more than three compared to a typical autoinjector comprising a syringe. This is advantageous for a user like a patient, because the distance from the skin to the position where the device is triggered is much less.

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir0); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

This patent application claims the priority of the European patent application 20315495.0, the disclosure content of which is hereby incorporated by reference.

### LIST OF REFERENCES

100 Autoinjector
105 Squeeze arrangement
110 Pouch
115 Pouch cross-section
120 Narrowing portion
125 Housing
130 Roller
140 Roller stopper
150 Torsion spring
160 Delivery tube
165 Spout
170 Plate
180 Needle
185 Needle opening
190 Trigger opening
195 Window
300 Needle drive mechanism
310 Drive spring
320 Trigger button
325 Trigger button main body
330 Trigger button arm
340 Needle holder
350 Needle holder ledge
360 Collar
365 Collar base
370 Collar spacing
380 Collar holding arm
390 Collar trigger arm
400 Base element
405 Base element main body
410 Base element arm
420 Base element spacing
Xro Rotation axis
Zrol Roller axis

1. Autoinjector (100) comprising
a housing (125) which is configured to receive a reservoir (120) with a fluid,
a squeeze arrangement (105), which is configured to drive the fluid from the reservoir (120) towards an outlet (180) of the autoinjector in a dispensing operation.

2. Autoinjector (100) according to claim 1, wherein the squeeze arrangement (105) comprises a movable element (130) which mechanically interacts with the reservoir (120) such that a movement of the movable element (130) causes a squeezing of the reservoir (120) to drive at least a portion of the fluid from the reservoir (120) towards the outlet (180).

3. Autoinjector (100) according to any of the claim 1 or 2, comprising a spring (150) comprising a spring portion which is configured to rotate around a rotation axis (Xro) and which is mechanically coupled to the movable element (130), such that it moves the movable element around the rotation axis (Xro).

4. Autoinjector (100) according to claim 3, wherein the spring (150) comprises a torsion spring (150) which is configured to move the movable element (130) around the rotation axis (Xro) such that the fluid contained in the reservoir (120) is moved towards the outlet within one revolution of the movable element (130) around the rotation axis (Xro) or less.

5. Autoinjector (100) according to any of the claims 1-4, wherein the reservoir (120) is arranged circumferentially around the rotation axis (Xro), wherein the reservoir is oriented along a circle segment, such that the fluid contained in the reservoir (110) can be driven towards the outlet (180) with a rotation of the movable element (130) which comprises less than a full revolution around the rotation axis (Xro).

6. Autoinjector (100) according to any of the claims 1-5, wherein the reservoir (120) comprises a collapsible pouch (120).

7. Autoinjector (100) according to any of the claims 1-6, wherein the housing (110) comprises a supporting element (170) in form of a protrusion protruding from a bottom of the housing (110) towards the reservoir (120), wherein the reservoir (120) is arranged between the supporting element (170) and the movable element (130) such that when the reservoir (120) is squeezed by the movable element (130) a surface of the reservoir (120) is pressed towards the supporting element (170).

8. Autoinjector (100) according to any of the claims 2-7, wherein the movable element (130) comprises a roller (130), which is configured to rotate around a roller axis (Zrol) while it is rotating at the same time around the rotation axis (Xro) and squeezes the reservoir (130).

9. Autoinjector according to claim 8, wherein the roller (130) comprises a shape of a cylinder or a cone.

10. Autoinjector (100) according to any of the claims 1-9, comprising a delivery tube (160) which is in fluid communication with the reservoir (120), and which is arranged between the reservoir (120) and the outlet (180) such that the delivery tube (160) supplements fluid to the outlet (180) which is provided by the reservoir (120) during a dispensing operation.

11. Autoinjector according to any of the preceding claims, comprising an outlet drive mechanism (300) comprising
an outlet (180),
an interface element (340) which is connected to or integrated with the outlet (180), wherein the interface element (340) is movable from a first axial position to a second axial position along the rotation axis (Xro),
a trigger (320) which is operatively connected to the interface element (350) wherein
the trigger (320) is movable from a first trigger position to a second trigger position along the rotation axis (Xro), wherein
in the first trigger position the interface element (340) is releasably locked from moving from the first axial position to the second axial position, and
in the second trigger position, the interface element (340) is movable to the second axial position, wherein
the movement of the trigger (320) from the first trigger position to the second trigger position causes the interface element (340) to be released from the first axial position such that the interface element (340) is movable to the second axial position.

12. Autoinjector according to claim 11, comprising a holding element (360) which is in mechanical contact with the interface element (340), wherein
the holding element (360) is rotatable around the rotation axis (Xro) relative to the interface element (340) from a blocking position to a release position, wherein
in the blocking position the interface element (340) is releasably locked by the holding element (360) to move from the first axial position to the second axial position, and
in the release position the interface element (340) is movable from the first axial position to the second axial position, wherein
the movement of the trigger (320) from the first trigger position to the second trigger position causes the holding element (360) to rotate from the blocking position to the release position.

13. Autoinjector according to claim 11 or 12, comprising an outlet drive unit (310) which is operatively connected to the trigger (320) and to the interface element (340), wherein
the outlet drive unit (310) being configured to provide energy for moving the interface element (340) from the first axial position to the second axial position, wherein the outlet drive unit (310) has a first drive unit state and a second drive unit state, wherein
in the first drive unit state the outlet drive unit (310) has energy stored and the interface element (340) is in the first axial position and the holding element (360) is in the blocking position and the interface element (340) is prevented from moving to the second axial position, and
in the second drive unit state the outlet drive unit (310) can transfer energy to the interface element (340) so that the interface element (340) is moved the first axial position to the second axial position along the rotation axis (Xro) when the holding element (360) is in the release position, wherein
the movement of the trigger (320) from the first trigger position to the second trigger position causes the outlet drive unit (310) to change from the first drive unit state to the second drive unit state.

14. Autoinjector (100) according to any of the preceding claims, wherein the autoinjector (100) comprises the reservoir (120) with a medicament or drug.

15. Autoinjector (100) according to any of the preceding claims, being a disposable or single-use device, for providing a single dose.
